(19) United States
(12) Patent Application Publication
(10) Pub. No.: US 2018/0263673 A1
(43) Pub. Date: Sep. 20, 2018

(54) Flexible Spine Plate

(71) Applicants: Catherine A. Mazzola, Franklin Lakes, NJ (US); Nicholas Breault, Bloomfield, CT (US)

(72) Inventors: Catherine A. Mazzola, Franklin Lakes, NJ (US); Nicholas Breault, Bloomfield, CT (US)

(21) Appl. No.: 15/821,094

(22) Filed: Nov. 22, 2017

Related U.S. Application Data
(60) Provisional application No. 62/425,212, filed on Nov. 22, 2016.

(51) Int. Cl. A61B 17/80; A61B 17/70; A61F 2/30; A61F 2/44; A61B 17/86

(52) U.S. Cl. CPC A61B 17/8085; A61B 17/7059; A61F 2/30749; A61F 2002/30385; A61B 17/86; A61F 2002/30382; A61F 2/442

(57) ABSTRACT

A flexible spine plate, including a first mounting portion defining at least one top attachment opening, a second mounting portion defining at least one bottom attachment opening and a plate mid-portion, wherein the first mounting portion and second mounting portion are separated by the plate mid-portion, and wherein the plate-mid portion is configured to flex in the Sagittal Plane.

### RELATED APPLICATIONS

This application is related to and claims the benefit of priority of the filing date of U.S. Provisional Patent Application Ser. No: 62/425,212, filed on Nov. 22, 2016, the contents of which are incorporated herein by reference in its entirety.

### FIELD OF THE INVENTION

This invention relates generally to a vertebral stabilization plate and more particularly to a vertebral stabilization plate which is flexible to allow a patient to have an increased range of mobility.

### BACKGROUND OF THE INVENTION

There are many conditions that require the use of a spinal plate to stabilize the spine of a patient in order to prevent additional damage from occurring. These conditions include trauma, burst fractures, tumors and disc degenerative conditions. These plates are typically used to address the instability of the vertebrae and typically involve using a fixation device to ‘fix’ adjacent vertebrae in one position relative to each other. These fixation devices typically include a spinous process plate (i.e. spinal plate) which extends between targeted vertebrae, wherein the spinal plate is affixed to the targeted vertebrae via percutaneous pedicle screws and/or facet screws. These current fixation devices provide for a rigid and/or a semi-rigid stabilization to limit the movement of the vertebrae of the patient. Unfortunately however, while these current fixation devices are semi-rigid they do not allow for flexible and more natural movement of the patient. This is undesirable because this restriction of natural movement may cause undue stress on the vertebrae above and below the targeted vertebrae to which the spinal plate is affixed.

### SUMMARY OF THE INVENTION

A flexible spine plate, including a first mounting portion defining at least one top attachment opening, a second mounting portion defining at least one bottom attachment opening and a plate mid-portion, wherein the first mounting portion and second mounting portion are separated by the plate mid-portion, and wherein the plate-mid portion is configured to flex in the Sagittal Plane

### BRIEF DESCRIPTION OF THE DRAWINGS

The foregoing and other features and advantages of the present invention will be more fully understood from the following detailed description of illustrative embodiments, taken in conjunction with the accompanying drawings in which like elements are numbered alike:

FIG. 1A illustrates a rear view of a flexible spine plate, in accordance with one embodiment of the invention.

FIG. 1B illustrates a front side perspective view of the spine plate of FIG. 1A.

FIG. 1C illustrates a side view of the spine plate of FIG. 1A being associated with the vertebra of a patient, in accordance with one embodiment of the invention.

FIG. 1D illustrates a rear view of a flexible spine plate, in accordance with another embodiment of the invention.

FIG. 1E illustrates a rear view of the flexible spine plate of FIG. 1A and a representation of the body planes.

FIG. 2A illustrates a rear view of a flexible spine plate, in accordance with another embodiment of the invention.

FIG. 2B illustrates a side view of the spine plate of FIG. 2A.

FIG. 2C illustrates a front side perspective view of the spine plate of FIG. 2A.

FIG. 2D illustrates a sides sectional view of the spine plate of FIG. 2A being associated with multiple (three) vertebra of a patient, in accordance with still yet another embodiment of the invention.

FIG. 3A illustrates a rear view of a flexible spine plate, in accordance with yet another embodiment of the invention.

FIG. 3B illustrates a side view of the spine plate of FIG. 3A.

FIG. 3C illustrates a front side perspective view of the spine plate of FIG. 3A.

FIG. 3D is a top side perspective view of an artificial disc for use with the flexible spine plate of FIG. 3A, in accordance with one embodiment of the invention.

FIG. 3E illustrates a side view of the spine plate of FIG. 3A being associated with vertebra of a patient and the artificial disc of FIG. 3D, in accordance with still yet another embodiment of the invention.

FIG. 4A illustrates a rear view of a flexible spine plate, in accordance with still yet another embodiment of the invention.

FIG. 4B illustrates a front side perspective view of the spine plate of FIG. 4A.

FIG. 4C is a top side perspective view of an artificial disc for use with the flexible spine plate of FIG. 4A, in accordance with another embodiment of the invention.

FIG. 5A illustrates a rear view of a flexible spine plate, in accordance with still yet embodiment of the invention.

FIG. 5B illustrates a front side perspective view of the spine plate of FIG. 5A.

FIG. 5C illustrates a side view of the spine plate of FIG. 5A being associated with vertebra of a patient and the artificial disc of FIG. 4C, in accordance with still yet another embodiment of the invention.

FIG. 6A illustrates front perspective view of a flexible spine plate, in accordance with still yet embodiment of the invention.

FIG. 6B illustrates a front side perspective view of an artificial disc for use with the spine plate of FIG. 6A.

FIG. 6C illustrates a side view of the the artificial disc of FIG. 6B securely associated with the spine plate of FIG. 6A, in accordance with still yet another embodiment of the invention.

FIG. 7A illustrates front perspective view of a flexible spine plate, in accordance with still yet embodiment of the invention.

FIG. 7B illustrates a front side perspective view of an artificial disc for use with the spine plate of FIG. 7A.

FIG. 7C illustrates a side view of the the artificial disc of FIG. 7B securely associated with the spine plate of FIG. 7A, in accordance with still yet another embodiment of the invention.

FIG. 8A illustrates front perspective view of a flexible spine plate, in accordance with still yet embodiment of the invention.

FIG. 8B illustrates a front side perspective view of an artificial disc for use with the spine plate of FIG. 8A.

FIG. 8C illustrates a side view of the the artificial disc of FIG. 6B securely associated with the spine plate of FIG. 8A, in accordance with still yet another embodiment of the invention.

FIG. 9A illustrates front perspective view of a flexible spine plate, in accordance with still yet embodiment of the invention.

FIG. 9B illustrates a front side perspective view of an artificial disc for use with the spine plate of FIG. 9A.

FIG. 9C illustrates a side view of the the artificial disc of FIG. 9B securely associated with the spine plate of FIG. 9A, in accordance with still yet another embodiment of the invention.

### DETAILED DESCRIPTION OF THE INVENTION

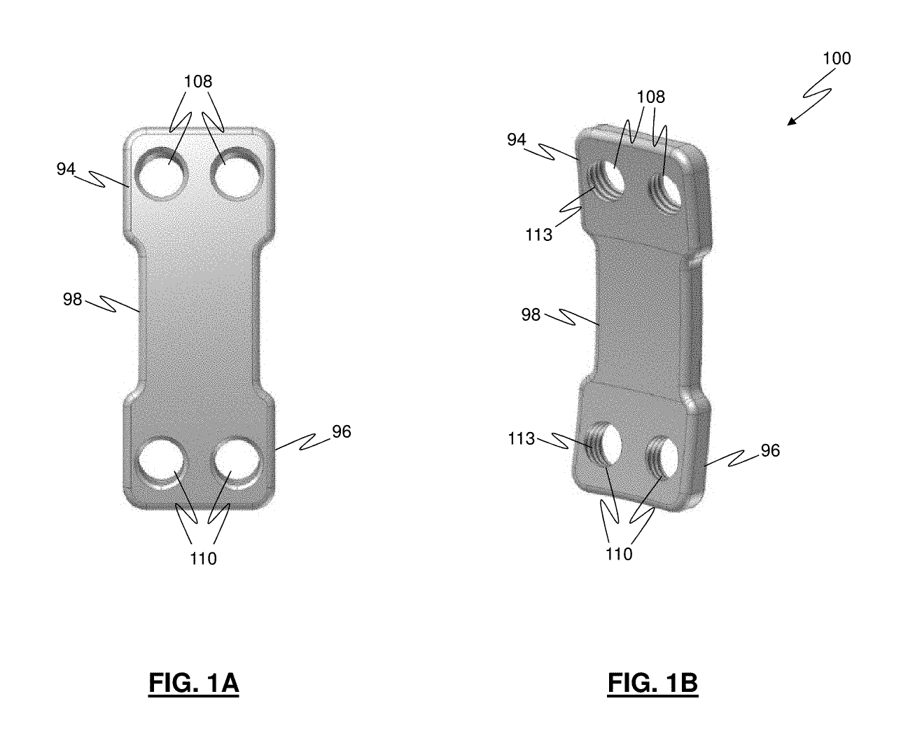

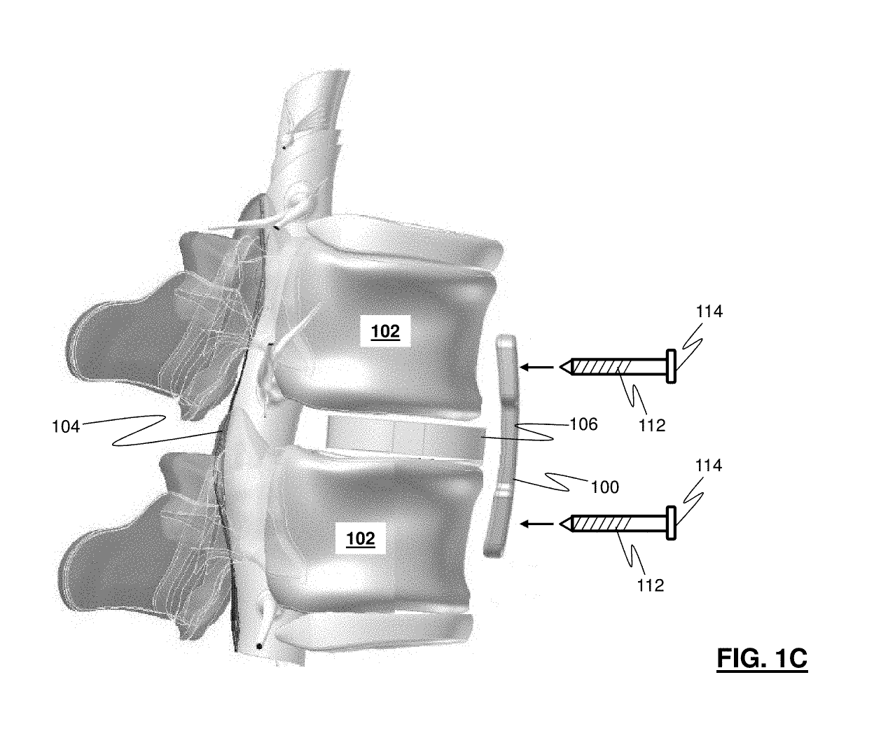

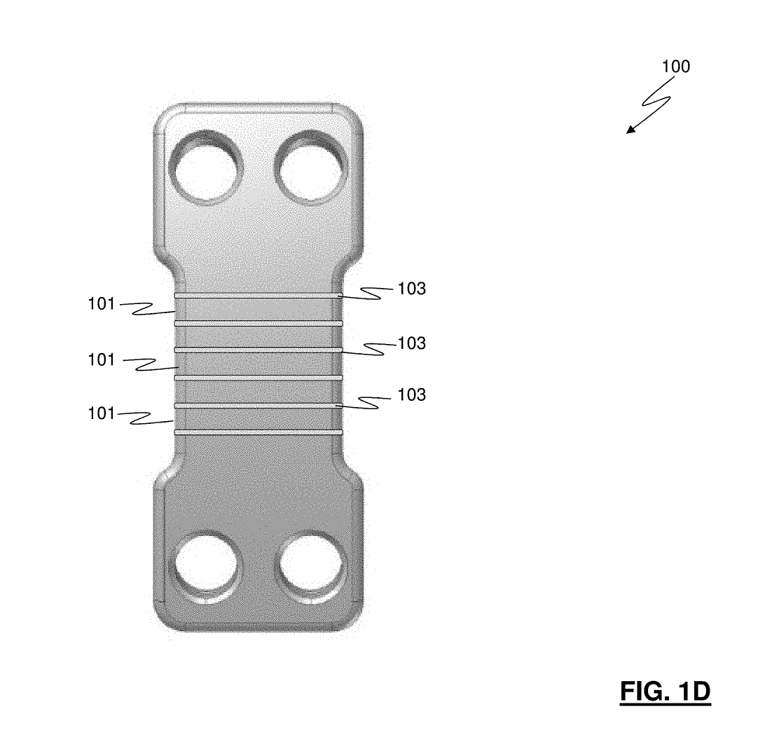

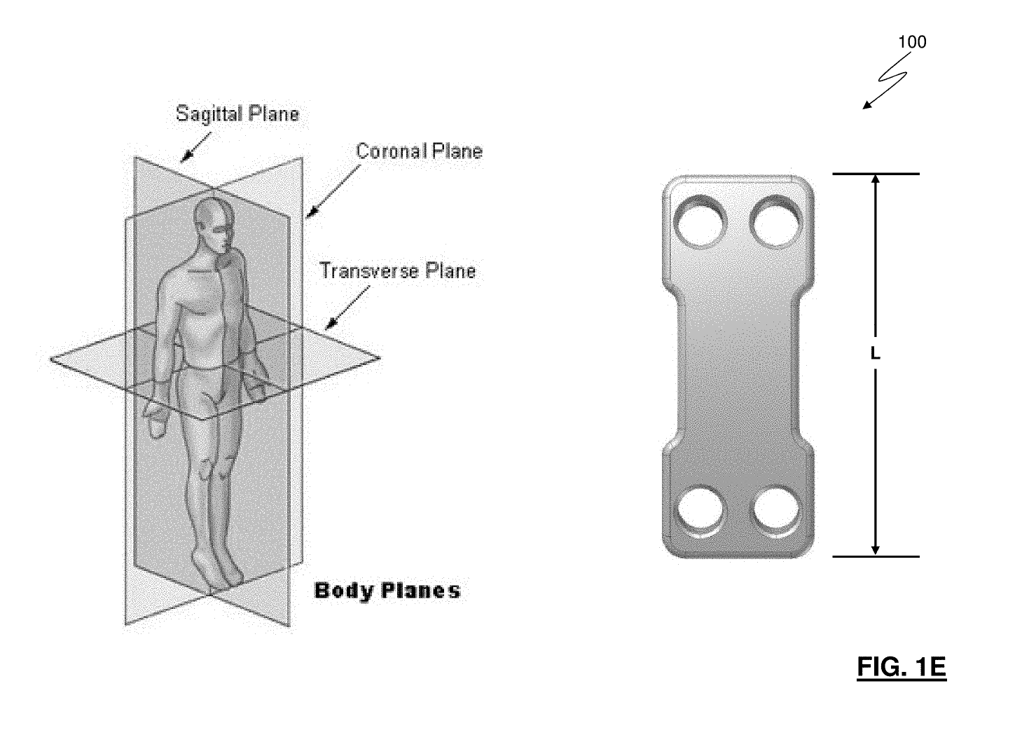

As disclosed herein with regards to an exemplary embodiment, referring to FIG. 1A and FIG. 1B, a flexible spine plate 100 is provided, wherein the spine plate 100 is configured to attach to vertebra 102 of the cervical and/or lumbar spine 104 of a patient. The spine plate 100 may be used and attached to the vertebra 102 after an artificial disc 106 is implanted between the patient's vertebra 102, wherein the flexible spine plate 100 may be attached to the vertebra 102 above and below where the patient's natural disc was removed and replaced with the artificial disc 106. The spine plate 100 includes a first mounting portion 94, a second mounting portion 96 and a plate mid-portion 98, wherein the first mounting portion 94 includes at least one top attachment opening 108 and the second mounting portion 96 includes at least one bottom attachment opening 110. The at least one top attachment opening 108 and the at least one bottom attachment opening 110 are configured to contain a mounting screw 114 having a mounting screw threaded portion 112, wherein the mounting screw threaded portion 112 is configured to securingly interact with the patient's vertebra 102. It should be appreciated that in one embodiment, the at least one top attachment opening 108 and/or at least one bottom attachment opening 110 may include a opening threaded portion 113 configured such that when the mounting screw 114 is located within the at least one top attachment opening 108 and at least one bottom attachment opening 110, the opening threaded portion 113 and the mounting screw threaded portion 112 threadingly and/or lockingly interact with each other.

Referring to FIG. 1C, the spine plate 100 may be attached to the vertebra 102 that is located above the artificial disc 106 via one or more mounting screws 114. Additionally, the spine plate 100 may be attached to the vertebra 102 that is located below the artificial disc 106 via one or more mounting screws 114. It should be appreciated that in one embodiment, the spine plate 100 may be manufactured using a flexible material that will allow the spine plate 100 to flex thereby allowing the vertebra 102 to maintain some, if not all, of its natural anatomic motion. It should be appreciated that in one embodiment, the plate mid-portion 98 may be constructed from a different material than the first mounting portion 94 and second mounting portion 96, such that the mid-plate portion 98 is less rigid than the first mounting portion 94 and second mounting portion 96. This would advantageously provide a more rigid mounting portion and allow the spine plate 100 to flex in the mid-plate portion 98.

Additionally, referring to FIG. 1D, in still yet another embodiment, it is contemplated that the mid-plate portion 98 may be constructed from multiple sections 101 that are held together via movable (i.e. living) hinges 103. In this embodiment, the hinges 103 may allow each of the multiple sections 101 to rotate a predetermined distance about the hinge 103. The hinges 103 may be configured to provide a predetermined amount of rotatable resistance, thereby allowing the mid-plate portion 98 to provide stability while allowing the patient to flex as desired. In still yet another embodiment, it is contemplated that the mid-plate portion 98 may be configured to be resilient to stretch in a lengthwise direction (approximately 10%) such that the length of the mid-plate portion 98 elongates in the direction of the first mounting portion 94 and second mounting portion 96. For example, in one embodiment, referring to FIG. 1E, the plate 100 includes a plate length L and the mid-plate portion 98 is at least partially constructed from a material that allows flexibility (approximately +/− 15 degrees) in the Sagittal Plane and/or Coronal Plane as well as flexibility along the plate length L. Additionally, this stretching may be accomplished via a material with a desired coefficient of elasticity or via hinges that telescope.

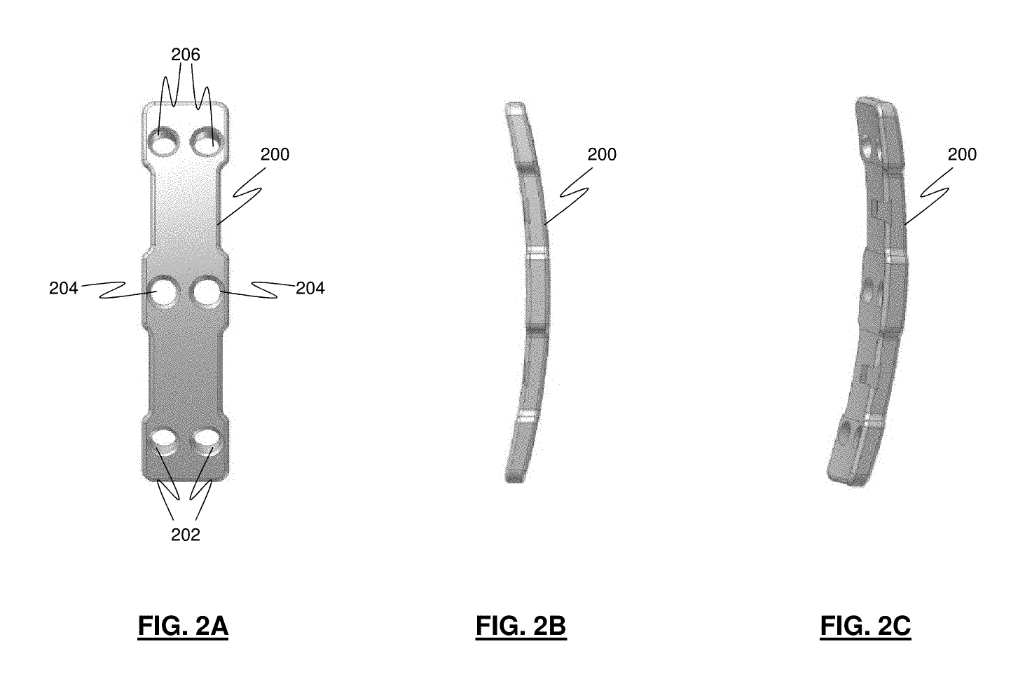

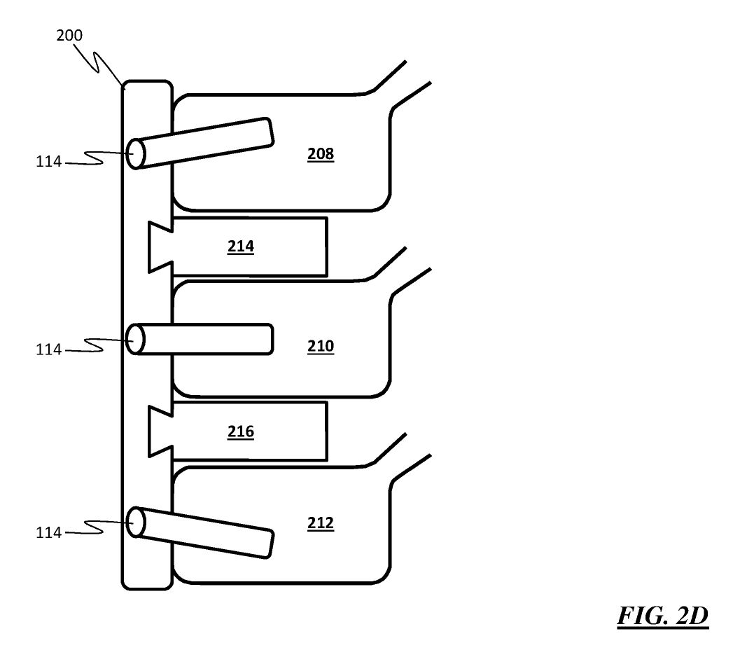

It should also be appreciated that the flexible spine plate 100 may be offered in a range of sizes for different applications and different sized patients, including single and/or multiple level disc replacements. Referring to FIG. 2A, FIG. 2B and FIG. 2C, a flexible spine plate 200 is provided which can accommodate a multiple level disc replacement (in this case two replacement discs), in accordance with another embodiment. In this embodiment, the flexible spine plate 200 includes at least one bottom attachment opening 202, at least one middle attachment opening 204 and at least one top attachment opening 206. Referring to FIG. 2D, the flexible spine plate 200 is configured to attach to a first vertebra 208, a second vertebra 210 and a third vertebra 212, wherein the first vertebra 208 is separated from the second vertebra 210 via a first disc 214 (which may be natural or artificial) and wherein the second vertebra 210 is separated from the third vertebra 212 via a second disc 216 (which may be natural or artificial).

Accordingly, the spine plate 200 may be attached to the first vertebra 208 that is above the first disc 214 via one or more mounting screws 114 associated with the at least one top attachment opening 206. Additionally, the spine plate 200 may be attached to the second vertebra 210 that is located below the first disc 214 via one or more mounting screws 114 associated with the at least one middle attachment opening 204 and the spine plate 200 may be attached to the third vertebra 212 that is located below the second disc 216 via one or more mounting screws 114 associated with the at least one bottom attachment opening 202. If the first disc 214 and/or second disc 216 are to be replaced then the first disc 214 and/or second disc 216 may be replaced with artificial discs and the spine plate 200 may be attached as described above. The placement and mounting of the spine plate 200 will act to keep the first disc 214 in place between the first vertebrae 208 and the second vertebrae 210 and/or the second disc 216 between the second vertebrae 210 and the third vertebrae 212.

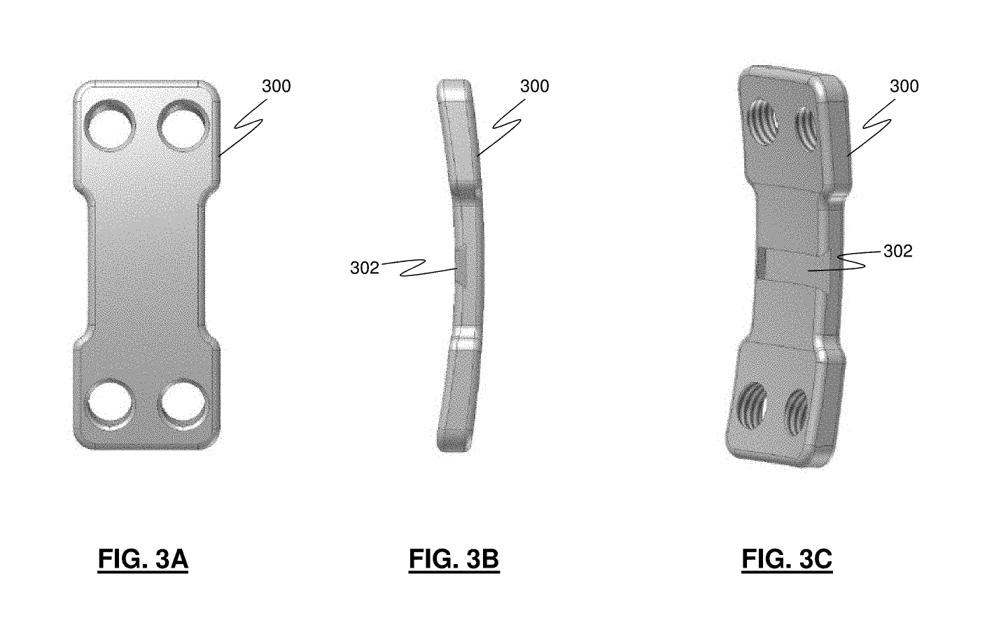

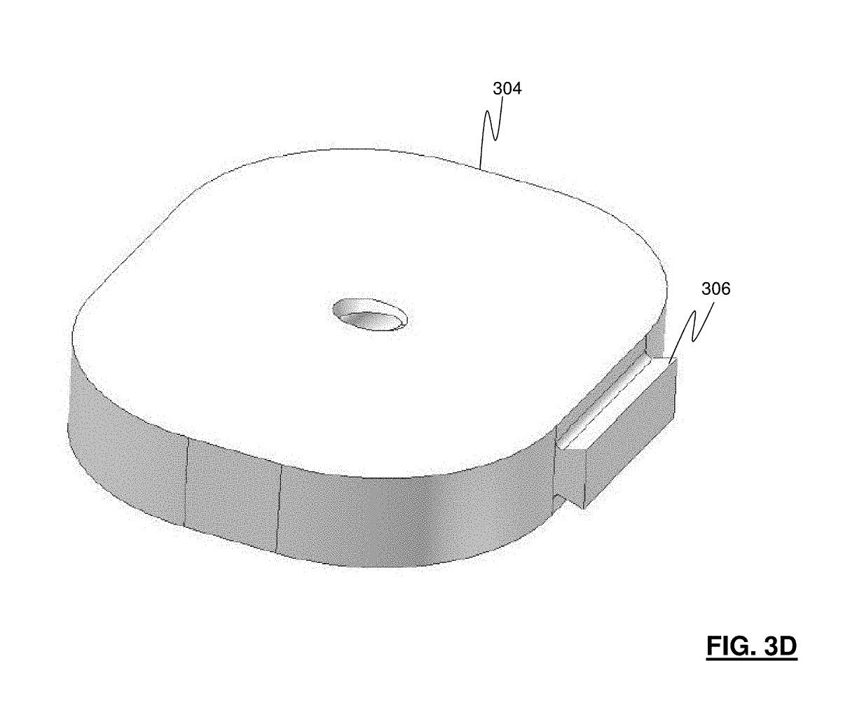

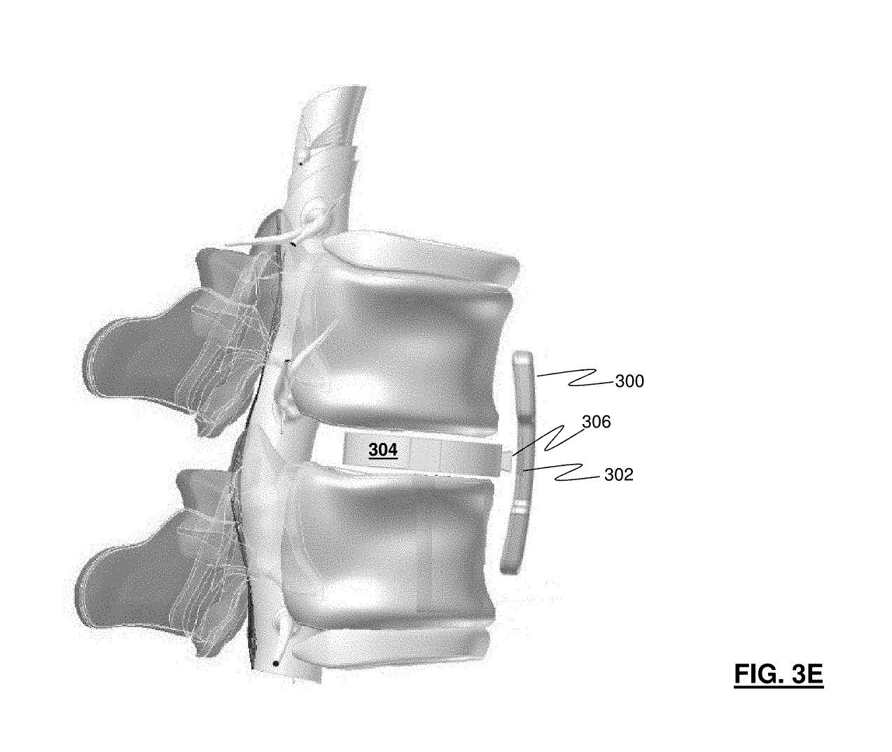

It should be appreciated that in other embodiments, it is contemplated that the artificial discs 106, 214, 216 and the spine plates 100, 200 may be configured to securely associate with each other. Referring to FIG. 3A, FIG. 3B, FIG. 3C and FIG. 3D, another embodiment of a spine plate 300 is provided, wherein the spine plate 300 defines a plate key cavity 302, wherein the plate key cavity 302 is configured to securely associate with an artificial disc 304 having a disc key protrusion 306. It should be appreciated that the disc key protrusion 306 is sized and shaped to be securely contained with the plate key cavity 302 when the artificial disc 304 is associated with the spine plate 300. In this embodiment, the artificial disc 304 is associated with the spine plate 300 by locating the disc key protrusion 306 within the plate key cavity 302 prior to placing the artificial disc 304 between adjacent vertebrae and attaching the spine plate 300 to the vertebra of the patient. It should be appreciated that in one embodiment, the plate key cavity or cut-out 302 may not traverse the entire width of the plate spine 300 thereby allowing the artificial disc 304 to stop and seat properly in a desired location on the spine plate 300. This advantageously allows both the artificial disc 304 and the spine plate 300 to engage each other and to lock together while being located as desired within the cavity between adjacent vertebrae.

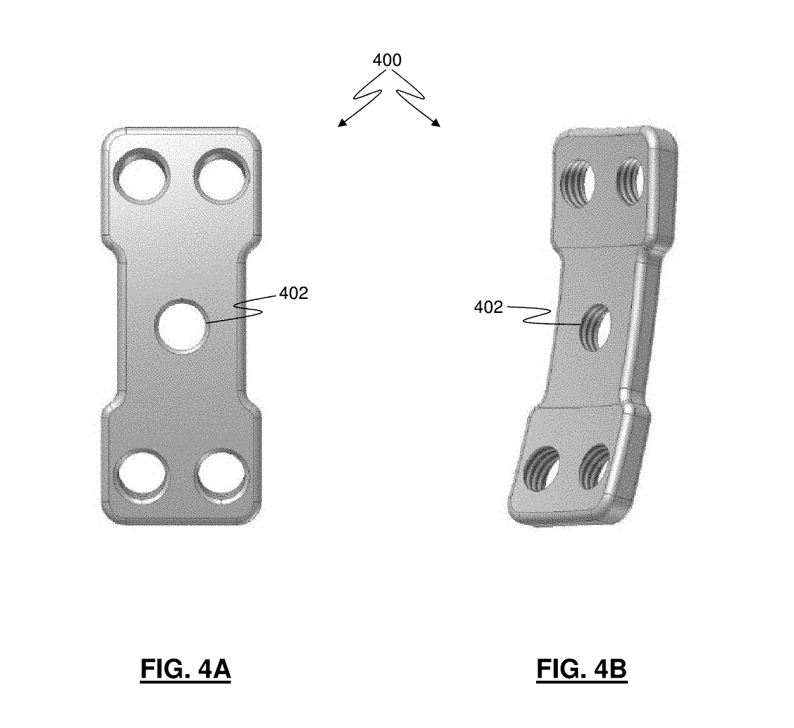

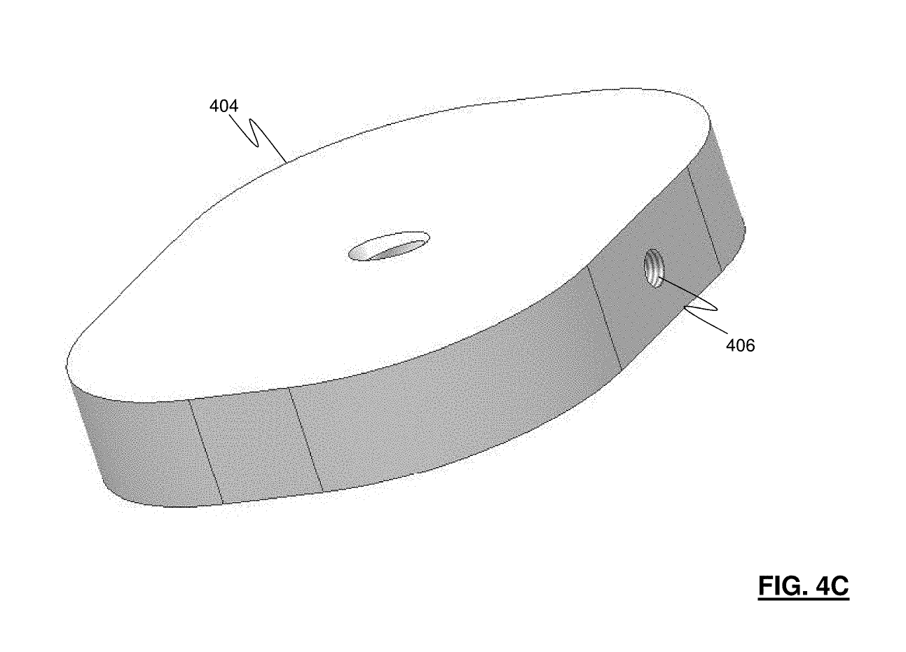

Referring to FIG. 4A, FIG. 4C and FIG. 4B, still yet another embodiment of a spine plate 400 is provided and defines a plate mounting cavity 402 (which may or may not be threaded), wherein the spine plate 400 is configured to securely associate with an artificial disc 404 which includes a threaded disc mounting cavity 406, wherein the threaded plate mounting cavity 402 and the threaded disc mounting cavity 406 are configured to securely contain a mounting screw that securely connects the spine plate 400 with the artificial disc 404.

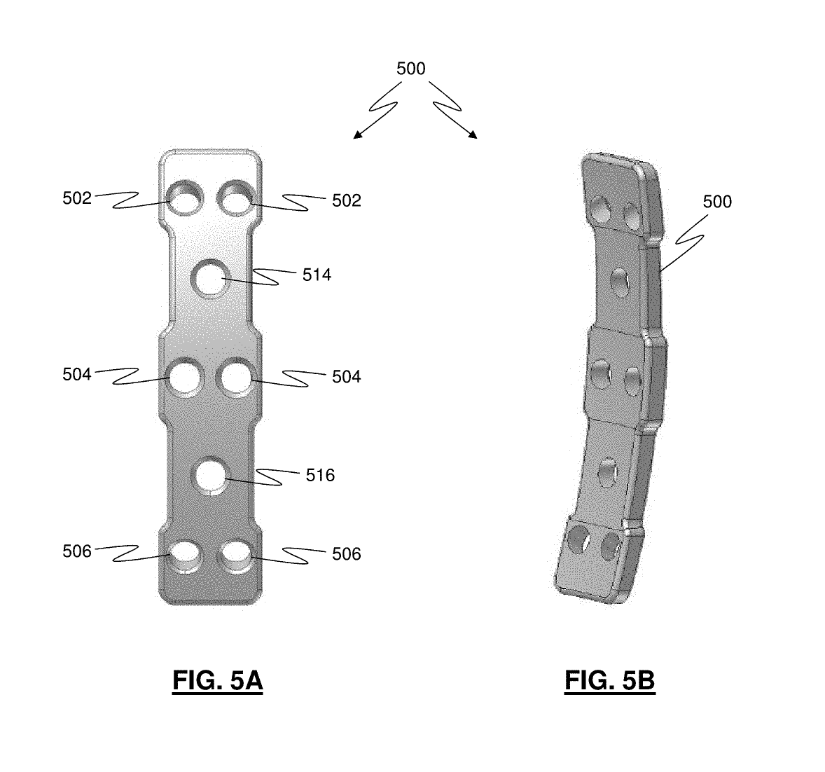

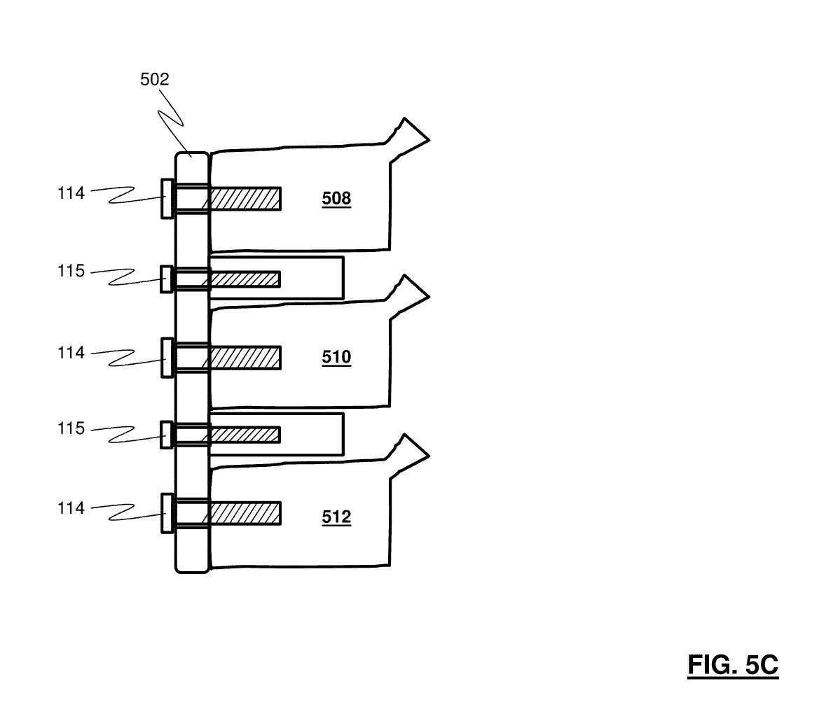

Additionally, it should be appreciated that in other embodiments, the spine plate 200 may also be configured with a plate key cavity 302 and/or a threaded plate mounting cavity 402 to securely associate with one or more replacement discs 304, 404. Referring to FIG. 5A, FIG. 5B and FIG. 5C, another embodiment of a spine plate 500 is shown, wherein the spine plate 500 is configured to securely associate with three (3) vertebrae. The spine plate 500 includes a first plate attaching opening 502, a second plate attaching opening 504 and a third plate attaching opening 506 to allow a mounting screw 114 to securely associate with a first vertebrae 508, a second vertebrae 510 and a third vertebrae 512. Additionally, the spine plate 500 includes a first plate mounting cavity 514 and a second plate mounting cavity 516 to allow the spine plate 500 to securely associate with the threaded disc mounting cavity 406 of the artificial disc 404 via a mounting screw 115 when the artificial disc 404 is located within the cavity between the first vertebrae 508 and the second vertebrae 510 and the cavity between the second vertebrae 510 and the third vertebrae 512. It is contemplated that in one embodiment, the spine plate 500 may be configured with a plate key cavity 302 to securingly associate with the artificial disc 304.

In another embodiment, it is contemplated that the first plate attaching opening 502, second plate attaching opening 504 and/or third plate attaching opening 506 may be angled allowing fixation of the artificial disc 404 to the vertebra 102 using traditional mounting screws 114. Additionally, in one embodiment the artificial disc 404 may have a hole for a fixation device (such as a screw) to attach it to the vertebrae. This would advantageously keep the artificial disc 404 from moving after it is inserted between the vertebrae. Moreover, the flexible spine plate 100, 200, 300, 400, 500 can be used independently of the artificial disc 404. One of the options, in addition to the multiple sizes, may be a flexible spine plate 100, 200, 300, 400, 500 without the mating cut out for use with the artificial disc 102. This will allow use of the spine plate 100, 200, 300, 400, 500 with other commercially available replacement discs.

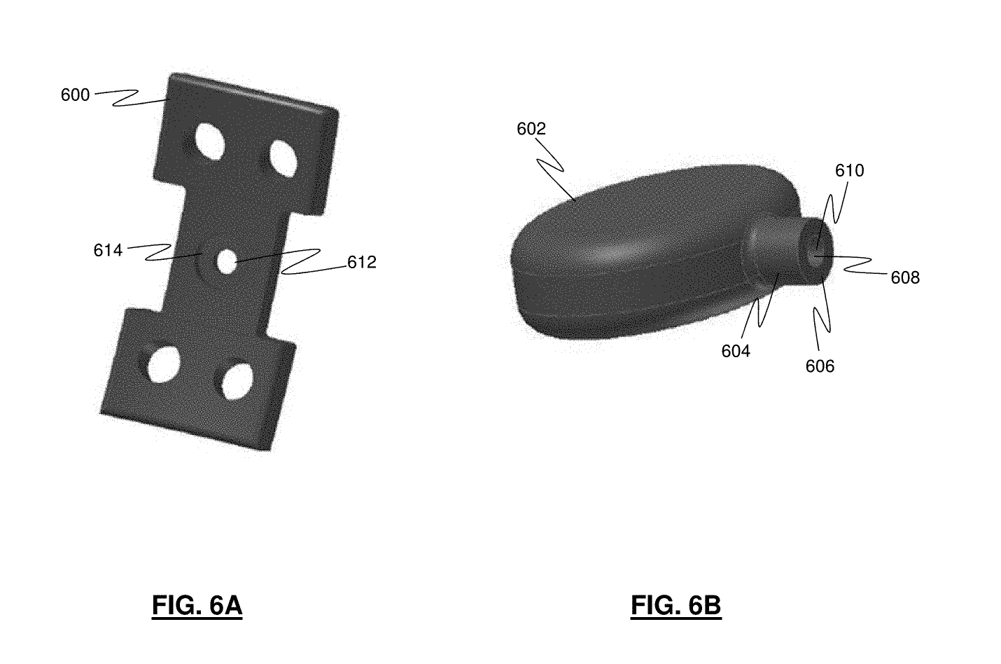

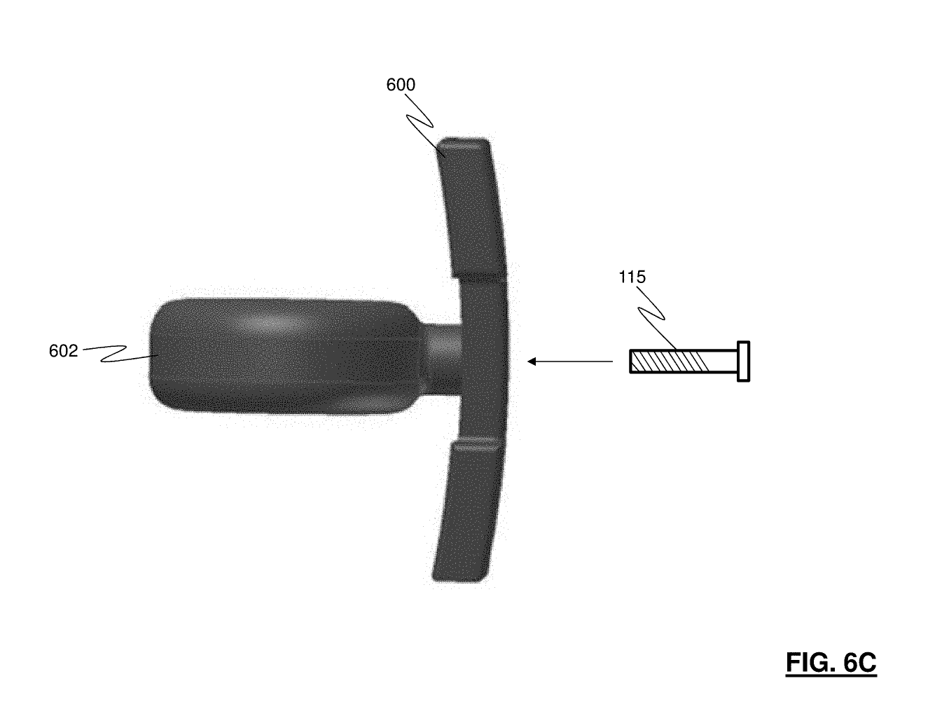

Referring to FIG. 6A, FIG. 6B and FIG. 6C, a spine plate 600 and an artificial disc 602 is shown in accordance with yet another embodiment of the invention. In this embodiment, the artificial disc 602 includes a mounting nub 604 having a mounting nub end 606 defining a nub cavity 608 having a threaded opening 610 for threadingly receiving and containing a mounting screw 114. Additionally, the spine plate 600 defines a disc mounting opening 612 having a countersunk portion 614, wherein the countersunk portion 614 is sized and shaped to interfacingly contain the mounting nub end 606. It should be appreciated that the spine plate 600 and the artificial disc 602 are securely associated by locating the mounting nub end 606 within the countersunk portion 614 and configuring the mounting screw 115 to extend through the disc mounting opening 612, through the threaded opening 610 and into the nub cavity 608.

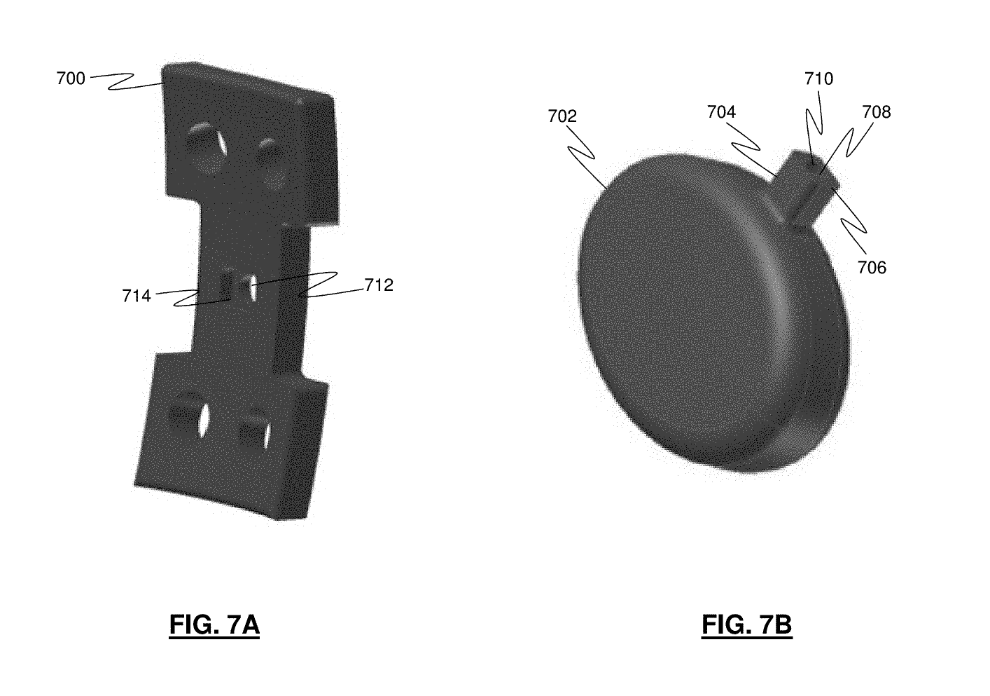

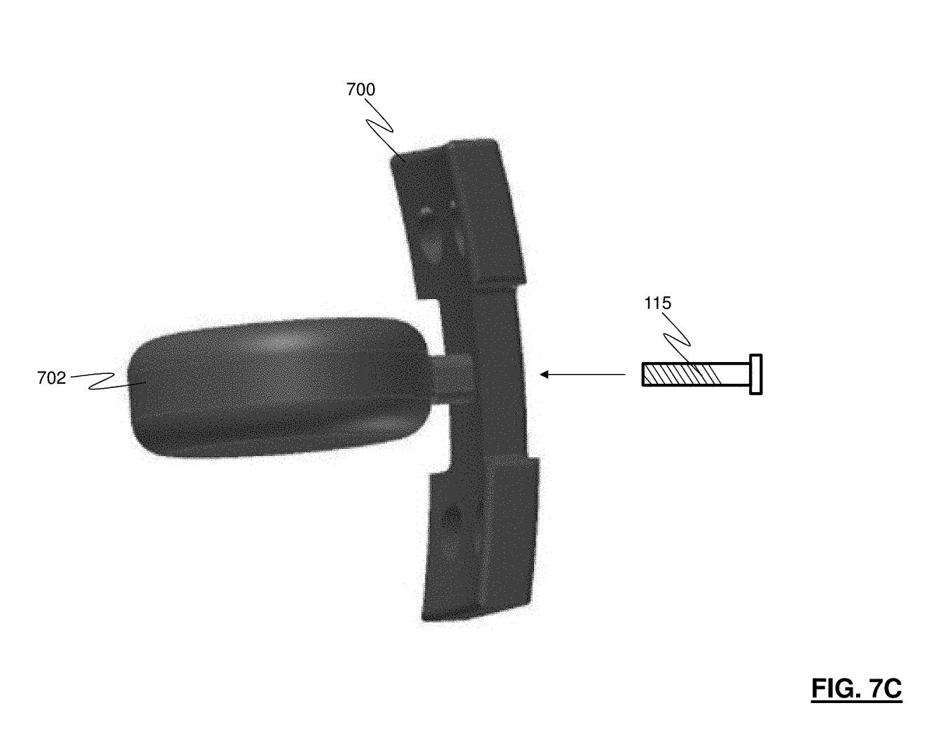

Referring to FIG. 7A, FIG. 7B and FIG. 7C, a spine plate 700 and an artificial disc 702 is shown in accordance with yet another embodiment of the invention. In this embodiment, the artificial disc 702 includes a mounting nub 704 having a mounting nub end 706 defining a nub cavity 708 having a threaded opening 710 for threadingly receiving and containing a mounting screw 115. Additionally, the spine plate 700 defines a disc mounting opening 712 having a countersunk portion 714, wherein the countersunk portion 714 is sized and shaped to interfacingly contain the mounting nub end 706. It should be appreciated that the spine plate 700 and the artificial disc 702 are securely associated by locating the mounting nub end 706 within the countersunk portion 714 and configuring the mounting screw 114 to extend through the disc mounting opening 712, through the threaded opening 710 and into the nub cavity 708. It should be preciated that the mounting nub 704 and/or mounting nub end 706 may be shaped to prevent rotation. For example, in this embodiment, the mounting nub 704 is rectangular in shape.

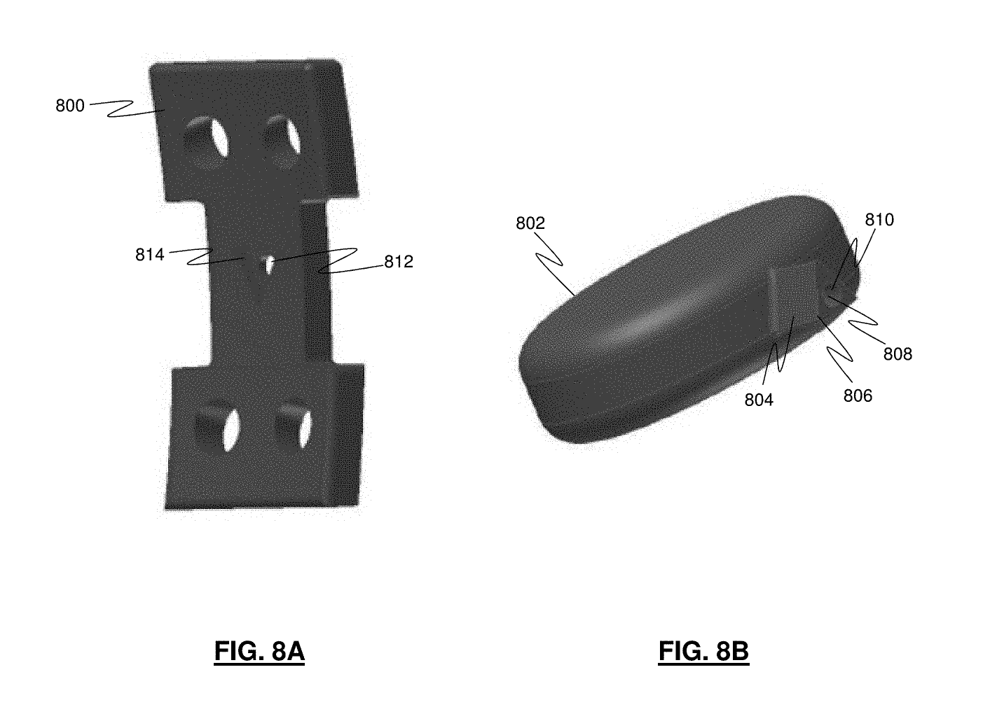

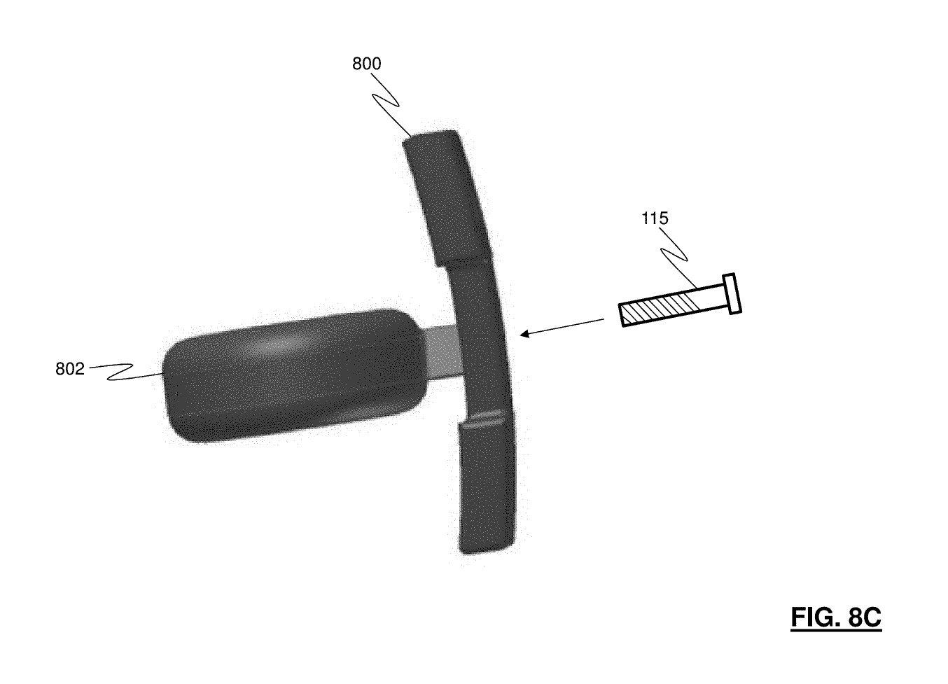

Referring to FIG. 8A, FIG. 8B and FIG. 8C, a spine plate 800 and an artificial disc 802 is shown in accordance with yet another embodiment of the invention. In this embodiment, the artificial disc 802 includes a mounting nub 804 having a mounting nub end 806 defining a nub cavity 808 having a threaded opening 810 for threadingly receiving and containing a mounting screw 115. Additionally, the spine plate 800 defines a disc mounting opening 812 having a countersunk portion 814, wherein the countersunk portion 814 is sized and shaped to interfacingly contain the mounting nub end 806. It should be appreciated that the spine plate 800 and the artificial disc 802 are securely associated by locating the mounting nub end 806 within the countersunk portion 814 and configuring the mounting screw 115 to extend through the disc mounting opening 812, through the threaded opening 810 and into the nub cavity 808. It should be appreciated that the mounting nub 804 and/or mounting nub end 806 may be shaped to prevent rotation. For example, in this embodiment, the mounting nub 804 is triangular in shape.

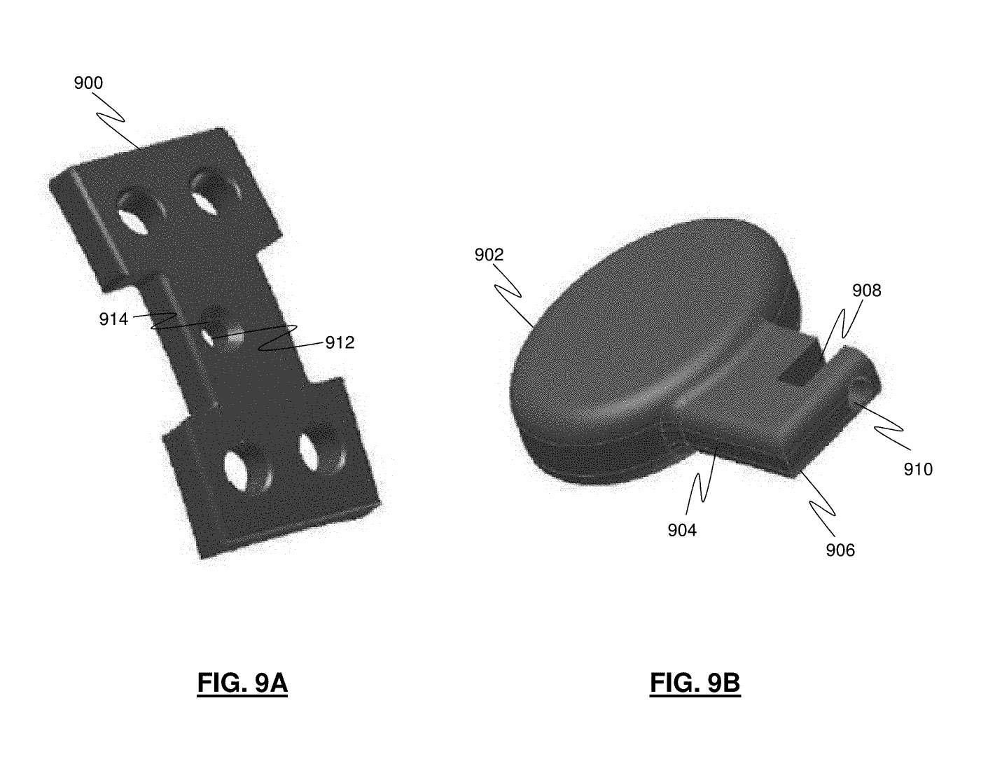

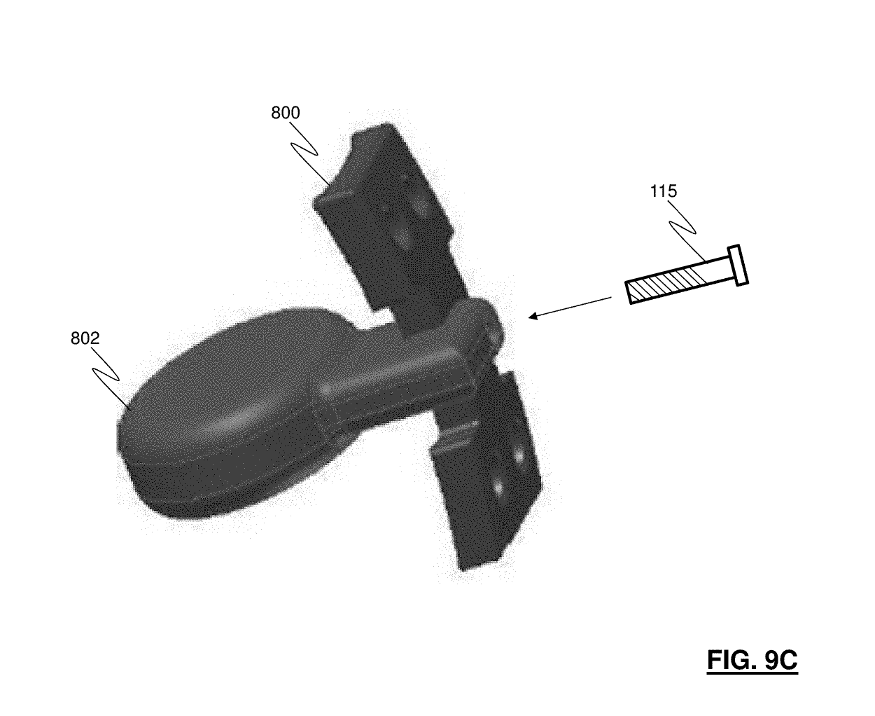

Referring to FIG. 9A, FIG. 9B and FIG. 9C, a spine plate 900 and an artificial disc 902 is shown in accordance with yet another embodiment of the invention. In this embodiment, the artificial disc 902 includes a mounting nub 904 having a mounting nub end 906 defining a nub cavity 908 having a threaded opening 910 for threadingly receiving and containing a mounting screw 115. Additionally, the spine plate 900 defines a disc mounting opening 912 for threadingly receiving and containing the mounting screw 115. It should be appreciated that the spine plate 900 and the artificial disc 902 are securely associated by locating the plate mid-portion 98 within the nub cavity 908 such that the disc mounting opening 912 is aligned with the threaded opening 910 and configuring the mounting screw 115 to extend through the threaded opening 910, into the nub cavity 908 and into the disc mounting opening 912. It should be appreciated that because the plate mid-portion 98 is located within the nub cavity 910 the disc 902 is prevented from rotating. It should be appreciated that disc mounting opening 612, 712, 812, 912 may or may not be configured to be threaded as desired.

It should be appreciated that in one embodiment the flexible spine plate 100, 200, 300, 400, 500 may include a plate mid-portion 98 which is thinner than the first mounting portion 94 and/or second mounting portion 96. This would advantageously allow the flexible spine plate 100, 200, 300, 400, 500 to controllably bend, wherein the force required to bend the flexible spine plate 100, 200, 300, 400, 500 may be responsive to the material and/or thickness of the plate mid-portion 98.

It should be appreciated that the flexible spine plate 100, 200, 300, 400 and/or artificial disc 106, 214, 216, 304, 404 may be constructed from any material or combination of materials as desired suitable to the desired end purpose. It is contemplated that in at least one embodiment, the material or combination of materials may have characteristics that allow flexibility to provide natural movement, not causing undue stress on the spinal levels above or below the plate, may allow and/or provide adequate support as well as flexibility, similar to a ligament, while performing a similar function as a ligament, may allow for flex, compression, tension, and other movements natural to the spine, may be constructed from a single material and/or may be constructed from bundles of thinly sliced materials attached together using a binder material to allow more rigid materials to flex, may be constructed from rigid materials perforated in such a way to provide adequate flexibility, may be constructed from meshes of intertwined materials, may be constructed from materials set in a matrix, may be constructed from a nano-material and/or nano-material combination(s), may be constructed from infused and impregnated materials, may be constructed from biomedical textiles, may be constructed from materials that stay in their plastic limits, allowing the material to move with the spine, but not to deform and/or may be constructed from materials with a memory of their original shape and will return to it after deformation.

It should be appreciated that some such material examples may include, but not be limited to Titanium, Polyetheretherketone (PEEK) (carbon fiber, hydroxyapatite, etc.), Stainless Steel, Cobalt Chromium, Platinum, Gold, Silver, Nitinol, Bone Cement, Aluminum, Ultrahigh molecular weight polyethylene (UHMWPE), Polyethylene (PE), Polyethylene terephthalate (PET or PETE), Nylon, Polyester, Acrylic, Polytetrafluoroethylene (PTFE), Ceramics, Plastics, Methyl methacrylate (MMA), Poly(methyl methacrylate) (PMMA), Metals and metal alloys, Polymers, Biological Materials, Synthetic Materials and/or Resorbable Materials.

Moreover, while the invention has been described with reference to an exemplary embodiment, it will be understood by those skilled in the art that various changes, omissions and/or additions may be made and equivalents may be substituted for elements thereof without departing from the spirit and scope of the invention. In addition, the elements and characteristics of the disclosed and/or contemplated embodiments may be combined in whole or in part and/or many modifications may be made to adapt a particular situation or material to the teachings of the invention without departing from the scope thereof. Accordingly, all of the information contained herein may be combined together (individually or wholly) or taken singly to achieve varying embodiments of the invention and to add to the scope of the invention without limiting the invention to a particular embodiment. Therefore, it is intended that the invention not be limited to the particular embodiment disclosed as the best mode contemplated for carrying out this invention, but that the invention will include all embodiments falling within the scope of the appended claims. Moreover, unless specifically stated any use of the terms first, second, etc. do not denote any order or importance, but rather the terms first, second, etc. are used to distinguish one element from another.

We claim:

1. A flexible spine plate, comprising:
a first mounting portion defining at least one top attachment opening;
a second mounting portion defining at least one bottom attachment opening; and
a plate mid-portion, wherein the first mounting portion and second mounting portion are separated by the plate mid-portion, and wherein the plate-mid portion is configured to flex in the Sagittal Plane.

2. The flexible spine plate of claim 1, wherein the at least one top attachment opening includes two top attachment openings configured to receive and contain a portion of a mounting device.

3. The flexible spine plate of claim 1, wherein the at least one bottom attachment opening includes two bottom attachment openings configured to receive and contain a portion of a mounting device.

4. The flexible spine plate of claim 1, wherein plate-mid portion is configured to flex approximately +/− 15 degrees in the Sagittal Plane.

5. The flexible spine plate of claim 1, wherein the plate-mid portion is configured to flex approximately +/− 15 degrees in the Coronal Plane.

6. The flexible spine plate of claim 1, wherein the plate-mid portion is configured to stretch approximately 10% lengthwise.

7. The flexible spine plate of claim 1, wherein the plate-mid portion defines a key cavity which extends across the plate mid-portion.

8. The flexible spine plate of claim 7, further includes an artificial vertebrae disc having a disc key protrusion, wherein the disc key protrusion is sized and shaped to be at least partially contained within the key cavity.

9. The flexible spine plate of claim 1, wherein the plate mid-portion defines at least one plate mounting cavity.

10. The flexible spine plate of claim 9, further includes an artificial vertebrae disc having a threaded disc mounting cavity, wherein the at least one plate mounting cavity is configured to be aligned with the threaded disc mounting cavity when the flexible spine plate and the artificial vertebrae disc are associated with each other.

11. The flexible spine plate of claim 1, wherein the plate-mid portion defines a key cavity configured to receive and contain a portion of an artificial vertebrae disc.
